(19) United States
(12) Patent Application Publication
(10) Pub. No.: US 2011/0117538 A1
(43) Pub. Date: May 19, 2011

(54) BIOREACTORS FOR FERMENTATION AND RELATED METHODS

(76) Inventor: Sarfaraz K. Niazi, Deerfield, IL (US)

(21) Appl. No.: 12/618,330

(22) Filed: Nov. 13, 2009

(51) Int. Cl. C12Q 1/70; C12M 1/12; C12Q 1/06

(52) U.S. Cl. 435/5; 435/297.1; 435/297.2; 435/39

(57) ABSTRACT

Bioreactors suitable for housing a predetermined volume of liquid comprising nutrient medium and biological culture comprising: (a) a container having at least one interior wall; (b) at least one nutrient medium inlet; (c) at least one liquid outlet; (d) at least one gas inlet; (e) at least one gas outlet; and (f) at least one cylindrical sparging filter attached to the at least one gas inlet, wherein the sparging filter comprises a plurality of pores along its axis which permit gas to be emitted radially from the sparging filter into the liquid, wherein the diameter of the plurality of pores does not exceed about 50 μm, and wherein the orientation of the at least one sparging filter within the container provides for immersion of the plurality of pores within the liquid and substantially uniform distribution of emitted gas throughout the liquid, and related methods of using said bioreactors to prepare various biological products.

### BACKGROUND OF THE INVENTION

A variety of vessels and methods have been developed over the years to carry out the fermentation of microorganisms, particularly bacteria and yeast, on a commercial scale. Stainless steel fermentation vessels of several hundreds of thousands liters are not uncommon, with the fermentation methods including batch, fed-batch, continuous or semi-continuous perfusion. The cells within these vessels are desirably kept in suspension, typically by rotating stirring blades located within the vessel, with gas exchange facilitated by the injection of air, oxygen or carbon dioxide into the vessel.

There are several drawbacks to this design. One is the introduction of shearing forces through the stirring blades and the cavitation of miniscule air bubbles, both being detrimental to more sensitive cell types or organisms. Also, these vessels should be rigorously cleaned between production runs to prevent cross-contamination, the latter being time consuming and requiring validation for individual cultures. Furthermore, the cost of stirred fermentors is relatively high on a volume basis, and thus these fermentors are commonly used over long periods of time. This, however, increases the risk of undesirable infection of mechanical failures. Perhaps most significantly, the optimization of culture conditions for stirred fermentors in a small scale cannot be transferred in a linear way to commercial scale production. For example, the fluid dynamics, aeration, foaming and cell growth properties change when the scale increases. In addition, for more delicate cell types or organisms, a large scale stirred fermentation vessel is not a viable device, even when more subtle stirring techniques such as airlift fermentors are used.

These drawbacks have led to the development of disposable fermentors. Examples of such disposable fermentors are systems based on wave agitation. See, e.g., U.S. Pat. No. 6,544,788; PCT Publication WO 00/66706. This type of fermentor may be used to culture relatively sensitive cells such as CHO cells (e.g., Pierce, Bioprocessing J. 3: 51-56 (2004)), hybridoma cells (e.g., Ling et al., Biotech. Prog., 19: 158-162 (2003)), insect cells (e.g., Weber et al., Cytotech. 38: 77-85 (2002)) and anchorage-dependent cells (e.g., Singh, Cytotech. 30: 149-158 (1999)) in a single disposable container. Such disposable units are relatively cheap, decrease the risk of infection because of their single use and require no internal stirring parts as the rocking platform upon which these containers reside during use induces wave-like forms in the internal liquid which facilitates gas exchange. However, this principle cannot be expanded to the size of hundreds of thousands of liters (such as the industrial fermentors) but are currently available from 1 liter to 500 liters (total volume of the disposable bag, available from Wave Biotechnology AG, Switzerland; Wave Biotech Inc., USA). Moreover, the hydrodynamics for each size of disposable bag will differ as a result of differences in depth and height. Therefore, the use of these disposable bags requires optimization and re-validation of each step in an up-scaling process.

Although bioreactor systems and related processes are known, improvements to such systems and processes would be useful in the preparation of a variety of products produced from a biological source.

### BRIEF SUMMARY OF THE INVENTION

The invention provides in one aspect a bioreactor suitable for use in preparing a variety of biological products. The bioreactor is suitable for housing a predetermined volume of liquid comprising nutrient medium and biological culture and comprises: (a) a container having at least one interior wall; (b) at least one inlet; (c) at least one outlet; (d) at least one gas inlet; (e) at least one gas outlet; and (f) at least one cylindrical sparging filter attached to the at least one gas inlet, wherein the sparging filter comprises a plurality of pores along its axis which permit gas to be emitted radially from the sparging filter into the liquid, wherein the diameter of the plurality of pores does not exceed about 50 μm, and wherein the orientation of the at least one sparging filter within the container provides for immersion of the plurality of pores within the liquid and substantially uniform distribution of emitted gas throughout the liquid.

A related aspect of the invention provides a method for producing a biological product from a predetermined volume of a liquid comprising nutrient medium and biological culture comprising (a) providing a bioreactor in accordance with the aforementioned aspect of the invention; (b) introducing nutrient medium and biological culture into the container; (c) passing gas through the sparging filter and into the liquid; (d) detecting the density of cells in the liquid at predetermined time intervals; and (e) removing the liquid and any biological product produced thereby from the container when the density of the cells in the liquid within the container reaches a predetermined value.

### BRIEF DESCRIPTION OF THE DRAWINGS

FIG. 1 is a side sectional view of a bioreactor in accordance with a preferred embodiment of the invention.

FIG. 2 is a side sectional view of a perfusion bioreactor in accordance with a preferred embodiment of the invention.

### DETAILED DESCRIPTION OF THE INVENTION

In one aspect, the present invention provides a bioreactor suitable for preparing a biological product from a predetermined volume of liquid comprising nutrient medium and biological culture, and a related method of use.

In one aspect, the bioreactor comprises: (a) a container having at least one interior wall; (b) at least one inlet for the nutrient medium and/or biological culture; (c) at least one outlet for liquid within the container; (d) at least one gas inlet; (e) at least one gas outlet; and (f) at least one cylindrical sparging filter attached to the at least one gas inlet, wherein the sparging filter comprises a plurality of pores along its axis which permit gas to be emitted radially from the sparging filter into the liquid within the container, wherein the diameter of the plurality of pores does not exceed about 50 μm, and wherein the orientation of the at least one sparging filter within the container provides for immersion of the plurality of pores within the liquid and for substantially uniform distribution of emitted gas throughout the liquid.

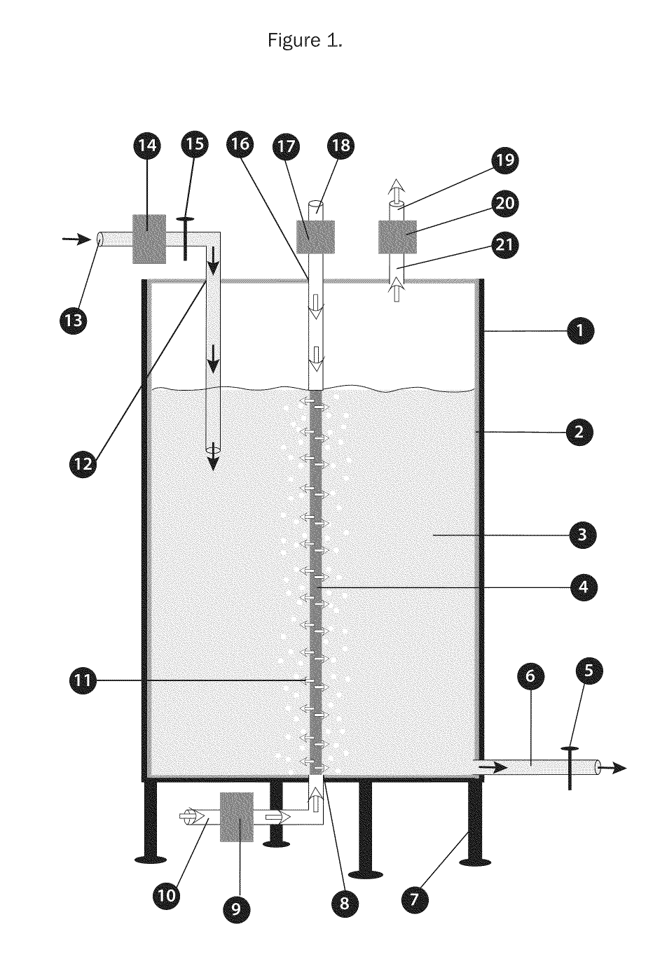

Turning initially to FIG. 1, a side sectional view of a preferred embodiment of the inventive bioreactor is illustrated. In this embodiment, there is provided a container 2 having at least one interior wall and, optionally, a support 1 for the container.

The container 2 provides a receptacle in which the liquid 3 comprised of nutrient medium and biological culture resides, and in which growth of the desired product occurs. A source of the nutrient medium and/or biological culture 13 is provided, with a filter 14 (when nutrient medium alone is fed into the container, e.g., during perfusion operation, as further described herein) and controllable valve 15 located upstream of the inlet 12. The nutrient medium is introduced into the container desirably via a tubular member. During operation, the end of this tubular member is immersed in the liquid.

A gas sparging filter 4 is provided within the container 2, the former being attached at one end, and desirably at both ends, to a container gas inlet 8, 16. Prior to entering the sparging filter, the gas, which is pressurized (compressed), passes from a gas source (10, 18) through a filter (9, 17), thus assisting in maintaining the sterility of the liquid within the container 3. After entering the interior of the sparging filter 4, the gas passes through the walls of the filter through the plurality of pores provided therein 11, and into the liquid 3 (as depicted by the arrows). The container 2 further includes a gas outlet 21 that is desirably fitted with a filter 20 upstream of the exhaust 19.

The container also includes a controllable valve 5 that controls removal of the liquid (nutrient media and/or biological culture from the container via an outlet 6, as desired, as well as an optional valve 15 for adding biological culture and/or nutrient media (commonly desired when operating the bioreactor in perfusion mode, as further described herein) into the container through an inlet 12 (via an optional filter 14) from a biological culture and/or nutrient media source 13.

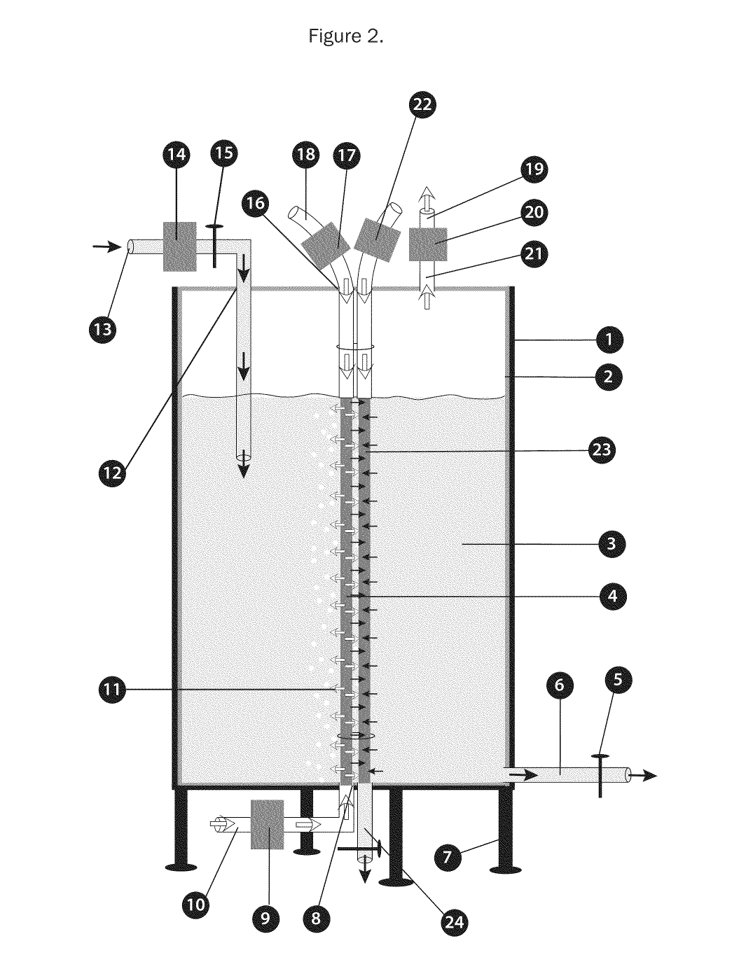

FIG. 2 illustrates an embodiment of the invention which is related to the embodiment depicted in FIG. 1. In FIG. 2, use of the bioreactor in perfusion mode is illustrated. Generally, a perfusion bioreactor retains biological culture in the container, with the product (e.g., a secreted product) being continuously withdrawn from the container and simultaneously replaced with an equivalent volume of nutrient media. In this illustrated embodiment, perfusion may be provided via a perforated cylinder 23 which retains the biological culture within the container, and a controllable outlet (desirably via a valve) 24 which permits removal of the product from the container.

Returning to FIG. 1, it is desirable that the container 2, particularly a flexible container as further described herein, be supported by a support 1, the latter preferably comprising platform 7 and side walls. The platform and side walls may be comprised of any suitable material, e.g., metal or rigid polymers, so long as it is sufficiently rigid to support the flexible container. Desirably, and as illustrated in FIG. 1, the platform (and the container) is raised relative to the floor or other surface. This permits inlets and outlets to be located on the side or the container which rests on the platform. For example, and as illustrated in this embodiment, it is desirable that the at least one gas inlet 8 of the container 2 be located on a portion of the container which is coextensive with the platform, wherein the platform includes an opening therethrough which permits the gas to pass through the platform and into the container 2 through the gas inlet 8.

Aspects of the present invention address various deficiencies in known bioreactor designs including, for example, maintaining a desirable level of suspension of the biological culture in the nutrient medium, and assuring proper aeration, each of which supports growth of the culture. Known bioreactors utilize are variety of means to provide adequate suspension and aeration including the use of impellers and/or movement of the container to effect mechanical circulation. In the inventive bioreactors, however, the interior of the container is free of mechanical agitation devices, such as impellers and the like, and the liquid within the container need not be moved via any mechanical means during operation of the bioreactor. Moreover, there is no need to shake or otherwise move the container during operation of the bioreactor; the bioreactor may remain stationary during operation.

The inventive bioreactor includes at least one cylindrical sparging filter attached to the at least one gas inlet, this filter comprising a plurality of pores along its axis which permit gas to be emitted radially from the sparging filter into the liquid. In this sparging filter, the mean diameter of each of the plurality of pores does not exceed about 50 μm, desirably from about 1 lam to about 50 μm, more desirably from about 1 μm to about 20 μm, and most desirably from about 1 μm to about 10 μm, wherein air bubbles in the aqueous liquid do not exceed about 500 μm. Air bubbles having a mean diameter of about 1000 μm or greater were found to exert damaging effects on certain biological cultures. Preferably, the mean pore diameter does not exceed about 10 μm, with the air bubble size not exceeding about 100 μm.

When in use, the sparging filter is orientation within the container so that the plurality of pores therein are fully immersed in the liquid, and the gas passes through and is emitted from the sparging filter to provide a substantially uniform distribution of the emitted gas throughout the liquid. Thus, the bioreactor is designed to promote maximum gas transfer into the liquid, with minimal movement of the biological culture, yet enough to maintain the culture in suspension. The use of a sparging filter which provides relatively small diameter air bubbles was found to provide sufficient gas transfer over a relatively long distance without producing significant damaging turbulence.

While the container may be of any suitable shape, e.g., cuboid or cylindrical, it is desirably generally cylindrical, wherein a central axis of the container is collinear with the axis of one of the cylindrical sparging filters. As the pores of the sparging filter are distributed along the axial length of the filter, desirably along at least 50%, 60%, 70% 80% or 90% of the filter length, and the filter is disposed within the container so as to provide full immersion of all pores within the liquid, desirably in a substantially vertical orientation, the bioreactor is able to emit a constant stream of relatively small bubbles from the core toward the periphery of the container. This arrangement was found to assist in reducing undesirable shear forces, and to increase yield, relative to known bioreactors. It is believed that, when the air flow into the sparging filter is optimized, a laminar shear is induced which is sufficient to move any metabolic products away from the biological culture cells without disturbing any clusters of such cells, thus maintaining optimal production of such products. This arrangement, which provides for relatively uniform aeration of the liquid, also has the benefit of minimizing stratification of the liquid typically seen in bioreactors wherein aeration is provided only at the base of the bioreactor, wherein overcrowding results in non-homogenous productivity.

As the relatively fine bubbles move toward the periphery of the container, the bubbles tend to coalesce and become relatively less effective in gas transfer. Thus, there is a relationship between the rate of gas emission into the liquid and the size of the container. Generally, the axis of a sparging filter should be no more than about 2 feet, desirably no more than about 1.5 feet, and preferably no more than about 1 foot, from another sparging filter and/or a container side wall. Thus, when a sparging filter having a mean pore size of no more than about 50 μm is used, the effective reach of gas bubbles emitted from the filter desirably will not exceed about 2 feet, more desirably about 1.5 feet, and preferably about 1 foot. The discovery of this relationship permits scaling of the batch size, e.g., when relatively large containers are used, a plurality of the cylindrical sparging filters may be used, relatively equally spaced across the cross-section of the container using the aforementioned spacing parameters. While greater spacing between spargers and container walls than that described above are possible, an increase in gas pressure is required to propel the bubbles through this relatively greater distance, which results in the air bubbles causing undesired turbulence within the liquid.

The compressed gas feeding the sparging filter may be oxygen, but also may be air; the inventive bioreactor is capable of using air with exceptional results. Moreover, the container may comprise one or two gas inlets. In the latter case, the gas may be fed into both ends of the sparging filter, this arrangement being illustrated in FIG. 1.

The sparger desirably comprises porous ceramic diffusers, which are commonly referred to as wicks or applicators. These materials permit precise control over porosity and pore size. These porous ceramics are manufactured by fusing aluminum oxide grains using a porcelain bond which provides a strong, uniformly porous and homogeneous structure capable of producing fine bubbles. Desirably, the porous ceramic may have 40-50% open porosity, and a pore size ranging from about 1 to about 90 microns (e.g., aluminum oxide porous ceramic is available in 6, 15, 30 and 50 micron pore sizes. The aforesaid ceramic may be of any suitable size, including but not limited to cylinders (tubes) from ½″ to 4″ diameter. Suitable porous ceramic diffusers are available from Refractron Technologies Corp. (New York).

Sparging may be run continuously, periodically, or in some cases, in response to certain events, e.g., within a bioreactor system and/or within an individual container. For example, the spargers may be connected to one or more sensors and a control system which is able to monitor the amount of sparging, the degree of foaming, the amount or concentration of a substance in the container, and respond by initiating, reducing, or increasing the degree of sparging of one or more compositions of gases.

As previously mentioned, the container desirably may include one or more sensors or probes for monitoring one or more process parameters inside the containers such as, for example, cell density, temperature, pressure, pH, dissolved oxygen (DO), dissolved carbon dioxide (DCO₂), mixing rate, and gas flow rate. The sensors for DO, pH and DCO₂ are desirably optical sensors, with the first two more desirably being disposable (e.g., TruFluor sensors, Finesse Solutions LLC, Santa Clara, Calif. or CellPhase sensors, Fluorometrix Corporation, Stow, Mass. 01775). Each sensor is intended to be in communication with a computer-implemented control system (e.g., a computer) for calculation and control of various parameters and for display and user interface. Such a control system may also include a combination of electronic, mechanical, and/or pneumatic systems to control the aforementioned processing parameters as required to stabilize or control the parameters (e.g., pH may be adjusted by the addition of CO₂ or ammonia). It should be appreciated that the control system may perform other functions and the invention is not limited to having any particular function or set of functions.

The one or more control systems described herein can be implemented in numerous ways, such as with dedicated hardware and/or firmware, using a processor that is programmed using microcode or software to perform the functions recited above or any suitable combination of the foregoing.

The processing device may also be in communication with various devices which can adjust the process parameters toward predetermined acceptable levels, for example, activating a heater, activating a gas inlet valve to adjust the oxygen or CO₂ levels, activating the gas outlet valve to reduce gas pressure in the headspace, and the like.

Advantageously, the bioreactor may further include a controllable heating element 51, desirably located between the upper surface of the platform and the lower portion of the container, but which may also be oriented around the side walls of the container or its support. This element, when activated, is able to increase the temperature of the liquid with the container to a level which is optimal for the particular biological culture therein. The heating element may comprise a heat exchanger, a closed loop water jacket, an electric heating blanket, or a Peltier heater. Other heaters for heating a liquid inside a vessel are known to those of ordinary skill in the art and may be used alone or in combination with the foregoing device. The heater may also include a sensor for detecting the temperature of the liquid inside the container, e.g., a thermocouple and/or a resistance temperature detector (RTD). The thermocouple may be operatively connected to a process control module to control temperature of the contents in the vessel. Optionally, a heat-conducting material may be embedded in the surface of the container to provide a heat transfer surface to overcome the insulating effect of the material used to form other portions of the container.

Optionally, cooling of the container may be provided by a closed loop water jacket cooling system, a cooling system mounted on the platform, or by standard heat exchange through a cover/jacket associated with the support, for example, a heat blanket or a packaged dual unit which provides heating and cooling may a component of a device configured for both heating/cooling but may also be separate from a cooling jacket. Cooling may also be provided by Peltier coolers.

In a related aspect, the bioreactor may be operated to provide for perfusion. In perfusion, the biological culture is placed into steady-state operation, thereby permitting operation of the bioreactor to be extended for weeks, and perhaps months. The perfusion bioreactor of the invention may be used to produce secreted products, produce large amounts of slow growing cells, or function as an artificial organ such as an extracorporeal liver. The design make this device ideal for hospital use in cell and gene therapy applications.

During perfusion, a liquid (the product, or byproducts) is removed from the bioreactor, while nutrients are introduced into the container periodically during operation, typically at a relatively slow rate, in order to maintain the volume of liquid therein reasonably constant. In the case of secreted products, this liquid desirably contains a product that requires purification. When the desired product is the culture itself, liquid containing toxic byproducts is removed during operation. Generally, one desires to prevent biological cultures from leaving the container during removal of the liquid therefrom; the present invention provides for this, as further described herein. In practice, a relatively small amount of cell loss (<10%) is tolerated in order to remove dead and dying cells and to promote a low level of regrowth.

One means for controlling the volume of liquid in the container is by controlling the weight of the container, or the entire bioreactor assembly, so that the weight remains constant. Desirably, a scale which determines the weight of the bioreactor provides feedback to a valve control which adjusts the valves associated with the container inlet and outlet to maintain the desired weight, and thus liquid volume in the container.

As shown in FIG. 2, a preferred embodiment of a perfusion bioreactor according to an aspect of the present invention comprises the bioreactor as described herein, with a perfusion filter. In FIG. 2, the perfusion function is performed as the media drains into perfusion filter 23 and exits the container under gravity, while a valve 25 may be used to control the rate of flow to a desired level. It should be appreciated that the perfusion bioreactor may have the same components as depicted and described in the bioreactor described herein, and as illustrated in FIG. 1. A valved, filtered inlet 14 for the additional of nutrient medium/culture from a source 13 into the container also is provided.

During operation, the container is at least partially filled with a liquid comprising nutrient media and biological cultures, sequentially or simultaneously. Oxygen, necessary for biological culture metabolism, is provided by air introduced into the container via the sparging filter 4. Exhaust gas is vented from the chamber through the gas outlet 21 and a downstream filter 20, which ensures that no biological cultures are released as an aerosol from the bioreactor. This further ensures that in the event of container depressurization, backflow through the vent 19 will not result in contamination of the container contents. The container is also provided with a culture/nutrient media inlet port 12 and a valved nutrient media outlet port 6.

While a perfusion filter of any design suitable for inclusion in the bioreactor described herein may be used, the perfusion filter 23 is desirably a perforated cylinder disposed within the liquid sufficiently close to the sparging filter to allow the relatively small air bubbles emitted from the sparging tube to provide a “scrubbing” function, whereby the biological cultures that might otherwise aggregate on the perfusion filter 23 are maintained in a continuously moving condition. Desirably, the perfusion filter is oriented substantially vertically within the container, with its axis substantially substantially parallel to the axis of the sparging filter, no more than about 12 inches, more desirably about 6 inches, even more desirably about 3 inches, even more desirably about 2 inches, and most desirably adjacent to (leaving a small gap to allow air bubbles to exit the sparger), the sparger. One skilled in the art should be able to determine the orientation of the perfusion filter relative to the sparger based on the description provided herein.

The perfusion filter may be comprised of any suitable material, e.g., stainless steel, ceramic, polymeric or cellulosic material, and have any porosity suitable to retain the culture residing in the container. In a preferred embodiment, the perfusion filter comprises a ceramic material, more preferably an aluminum oxide ceramic material, and has a mean pore size ranging from about 1 μm to about 100 μm, more preferably from about 1 μm to about 10 μm and more preferably about 6 μm. The filter is oriented with the container so the pores remain immersed within the liquid during operation of the bioreactor.

If desired, a plurality of perfusion filter may be provided, this being a particularly useful method of increasing the rate of removal of liquid product from the container. If multiple perfusion filters are used, they are preferably oriented around the sparging filter(s) as described herein. For example, three perfusion filters may be spaced equidistant around a single sparging filter, as necessary to obtain the desired product output.

The use of the present invention contemplates the use of pre-validated (and, preferably, pre-sterilized) containers, allowing one to provide for the production of biological products without the need for re-validation of the bioreactor.

One of the advantages of the inventive bioreactor and related method is the increase in product yield per container volume that may be obtained relative to known systems. Contributing to this increase in yield is the capability of the bioreactor to operate when the amount of liquid in each container exceeds 50 vol. %, based on the interior volume of the containers. Desirably, the amount of liquid in each container during operation of the bioreactor may exceed about 60 vol. % of the interior volume of the container, more desirably it may exceed about 70 vol. %, even more desirably it may exceed about 80 vol. %, it may preferably exceed about 85 vol. %, and more preferably it may exceed about 90 vol. % thereof. This increase in liquid volume on a percentage basis not only provides relatively high yields per volume, but may be achieved even if the liquid initially introduced into the bioreactor, or as present in the bioreactor during and/or after processing, contains relatively low levels of anti-foaming agents such as Antifoam 2210 or Compound A (Dow Corning), M-10 (Calgene), Breox FMT 30 International Specialty Company), or A6582, A6457, A6707, A8082 and A8582 (Sigma Aldrich)(from about 0.001 wt. % to about 0.005 wt. %). More desirably, the liquid initially introduced into the bioreactor, or as present in the bioreactor during and/or after processing, is substantially free of such anti-foaming agents (from about 0.0001 wt. % to about 0.001 wt. %, or less than about 0.001 wt %).

Each container is provided with a gas outlet which includes a pressure valve and submicron filter, the former assisting in maintaining the pressure within the container at a desired range while the latter assists in maintaining sterility of the liquid. Desirably, the pressure in the container is maintained at ambient conditions, preferably ranging from about to about 0.1 to 1 psig. The filter may be of any suitable size and porosity, but is preferably a HEPA filter, having an average porosity of from about 0.3 μm to about 0.1 μm, and more preferably of about 0.22 μm. Gas entering the container through a gas inlet also are desirably subjected to filtration by such filter element.

It is further desirable that the container be located within a climate-controlled environment. More desirably, the containers reside within a chamber which permits independent control of one or more of the temperature of the ambient air within the enclosure, of the air quality, and of the radiation to which the containers are exposed. Preferably, the environment permits the independent control of the ambient temperature, air quality and radiation for each container.

Generally, the invention provides bioreactors and methods which are universal in the sense that the invention is suitable and adaptable for processing a variety of compositions, including both biologic and non-biologic components. Indeed, an inventive bioreactor designed for use with mammalian cells, for example, may be used for culturing bacteria, allowing ease of manufacturing.

As used herein, the term “liquid” is intended to encompass compositions which include biologic components and nutrient medium as described herein.

Compositions comprising non-biologic components include, but are not limited to, those which comprise microcarriers (e.g., polymer spheres, solid spheres, gelatinous particles, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, cells, capillaries, and aggregates (e.g., aggregates of cells).

The biological components that may be processed in accordance with the invention are described in the paragraphs which follow and include, but are not limited to, cell cultures derived from sources such as animals (e.g., hamsters, mice, pigs, rabbits, dogs, fish, shrimp, nematodes, and humans), insects (e.g., moths and butterflies), plants (e.g., algae, corn, tomato, rice, wheat, barley, alfalfa, sugarcane, soybean, potato, lettuce, lupine, tobacco, rapeseed (canola), sunflower, turnip, beet cane molasses, seeds, safflower, and peanuts), bacteria, fungi, and yeast, as well as baculoviruses.

Illustrative animal cells include Chinese hamster ovary (CHO), mouse myeloma, M0035 (NSO cell line), hybridomas (e.g., B-lymphocyte cells fused with myeloma tumor cells), baby hamster kidney (BHK), monkey COS, African green monkey kidney epithelial (VERO), mouse embryo fibroblasts (NIH-3T3), mouse connective tissue fibroblasts (L929), bovine aorta endothelial (BAE-1), mouse myeloma lymphoblastoid-like (NSO), mouse B-cell lymphoma lymphoblastoid (WEHI 231), mouse lymphoma lymphoblastoid (YAC 1), mouse fibroblast (LS), hepatic mouse (e.g., MC/9, NCTC clone 1469), and hepatic rat cells (e.g., ARL-6, BRL3A, H4S, Phi 1 (from Fu5 cells)).

Illustrative human cells include retinal cells (PER-C6), embryonic kidney cells (HEK-293), lung fibroblasts (MRC-5), cervix epithelial cells (HELA), diploid fibroblasts (WI38), kidney epithelial cells (HEK 293), liver epithelial cells (HEPG2), lymphoma lymphoblastoid cells (Namalwa), leukemia lymphoblastoid-like cells (HL60), myeloma lymphoblastoid cells (U 266B1), neuroblastoma neuroblasts (SH-SY5Y), diploid cell strain cells (e.g., propagation of poliomyelitis virus), pancreatic islet cells, embryonic stem cells (hES), human mesenchymal stem cells (MSCs, which can be differentiated to osteogenic, chondrogenic, tenogenic, myogenic, adipogenic, and marrow stromal lineages, for example), human neural stem cells (NSC), human histiocytic lymphoma lymphoblastoid cells (U937), and human hepatic cells such as WRL68 (from embryo cells), PLC/PRF/5 (i.e., containing hepatitis B sequences), Hep3B (i.e., producing plasma proteins: fibrinogen, alpha-fetoprotein, transferrin, albumin, complement C3 and/or alpha-2-macroglobulin), and HepG2 (i.e., producing plasma proteins: prothrombin, antithrombin III, alpha-fetoprotein, complement C3, and/or fibrinogen).

Cells from insects (e.g., baculovirus and Spodoptera frugiperda ovary (Sf21 cells produce Sf9 line)) and cells from plants or food, may also be cultured in accordance with the invention. Cells from sources such as rice (e.g., Oryza sativa, Oryza sativa cv Bengal callus culture, and Oryza sativa cv Taipei 309), soybean (e.g., Glycine max cv Williams 82), tomato (Lycopersicum esculentum cv Seokwang), and tobacco leaves (e.g., Agrobacterium tumefaciens including Bright Yellow 2 (BY-2), Nicotiana tabacum cv NT-1, N. tabacum cv BY-2, and N. tabacum cv Petite Havana SR-1) are illustrative examples.

Bacteria, fungi, or yeast may also be cultured in accordance with the invention. Illustrative bacteria include Salmonella, Escherichia coli, Vibrio cholerae, Bacillus subtilis, Streptomyces, Pseudomonas fluorescens, Pseudomonas putida, Pseudomonas sp, Rhodococcus sp, Streptomyces sp, and Alcaligenes sp. Fungal cells can be cultured from species such as Aspergillus niger and Trichoderma reesei, and yeast cells can include cells from Hansenula polymorpha, Pichia pastoris, Saccharomyces cerevisiae, S. cerevisiae crossed with S. bayanus, S. cerevisiae crossed with LAC4 and LAC1-2 genes from K. lactis, S. cerevisiae crossed with Aspergillus shirousamii, Bacillus subtilis, Saccharomyces diastasicus, Schwanniomyces occidentalis, S. cerevisiae with genes from Pichia stipitis, and Schizosaccharomyces pombe.

A variety of different products may also be produced in accordance with the invention. Illustrative products include proteins (e.g., antibodies and enzymes), vaccines, viral products, hormones, immunoregulators, metabolites, fatty acids, vitamins, drugs, antibiotics, cells, hydrodomas, and tissues. Non-limiting examples of proteins include human tissue plasminogen activators (tPA), blood coagulation factors, growth factors (e.g., cytokines, including interferons and chemokines), adhesion molecules, Bc1-2 family of proteins, polyhedrin proteins, human serum albumin, scFv antibody fragment, human erythropoietin, mouse monoclonal heavy chain 7, mouse IgG_{2b,k}, mouse IgG1, heavy chain mAb, Bryondin 1, human interleukin-2, human interleukin-4, ricin, human α1-antitrypisin, biscFv antibody fragment, immunoglobulins, human granulocyte, stimulating factor (hGM-CSF), hepatitis B surface antigen (HBsAg), human lysozyme, IL-12, and mAb against HBsAg. Examples of plasma proteins include fibrinogen, alpha-fetoprotein, transferrin, albumin, complement C3 and alpha-2-macroglobulin, prothrombin, antithrombin III, alpha-fetoprotein, complement C3 and fibrinogen, insulin, hepatitis B surface antigen, urate oxidase, glucagon, granulocyte-macrophage colony stimulating factor, hirudin/desirudin, angiostatin, elastase inhibitor, endostatin, epidermal growth factor analog, insulin-like growth factor-1, kallikrein inhibitor, α1-antitrypsin, tumor necrosis factor, collagen protein domains (but not whole collagen glycoproteins), proteins without metabolic byproducts, human albumin, bovine albumin, thrombomodulin, transferrin, factor VIII for hemophilia A (i.e., from CHO or BHK cells), factor VIIa (i.e., from BHK), factor IX for hemophilia B (i.e., from CHO), human-secreted alkaline phosphatase, aprotinin, histamine, leukotrienes, IgE receptors, N-acetylglucosaminyltransferase-III, and antihemophilic factor VIII.

Enzymes may be produced from a variety of sources using the invention. Non-limiting examples of such enzymes include YepACT-AMY-ACT-X24 hybrid enzyme from yeast, Aspergillus oryzae α-amylase, xylanases, urokinase, tissue plasminogen activator (rt-PA), bovine chymosin, glucocerebrosidase (therapeutic enzyme for Gaucher's disease, from CHO), lactase, trypsin, aprotinin, human lactoferrin, lysozyme, and oleosines.

Vaccines also may be produced using the invention. Non-limiting examples include vaccines for prostate cancer, human papilloma virus, viral influenza, trivalent hemagglutinin influenza, AIDS, HIV, malaria, anthrax, bacterial meningitis, chicken pox, cholera, diphtheria, haemophilus influenza type B, hepatitis A, hepatitis B, pertussis, plague, pneumococcal pneumonia, polio, rabies, human-rabies, tetanus, typhoid fever, yellow fever, veterinary-FMD, New Castle's Disease, foot and mouth disease, DNA, Venezuelan equine encephalitis virus, cancer (colon cancer) vaccines (i.e., prophylactic or therapeutic), MMR (measles, mumps, rubella), yellow fever, Haemophilus influenzae (Hib), DTP (diphtheria and tetanus vaccines, with pertussis subunit), vaccines linked to polysaccharides (e.g., Hib, Neisseria meningococcus), Staphylococcus pneumoniae, nicotine, multiple sclerosis, bovine spongiform encephalopathy (mad cow disease), IgG1 (phosphonate ester), IgM (neuropeptide hapten), SIgA/G (Streptococcus mutans adhesin), scFv-bryodin 1 immunotoxin (CD-40), IgG (HSV), LSC(HSV), Norwalk virus, human cytomegalovirus, rotavirus, respiratory syncytial virus F, insulin-dependent autoimmune mellitus diabetes, diarrhea, rhinovirus, herpes simplex virus, and personalized cancer vaccines, e.g., for lymphoma treatment (i.e., in injectable, oral, or edible forms). Recombinant subunit vaccines also may be produced, such as hepatitis B virus envelope protein, rabies virus glycoprotein, E. coli heat labile enterotoxin, Norwalk virus capsid protein, diabetes autoantigen, cholera toxin B subunit, cholera toxin B an dA2 subunits, rotavirus enterotoxin and enterotoxigenic E. coli, fimbrial antigen fusion, and porcine transmissible gastroenteritis virus glycoprotein S.

Viral products also may be produced. Non-limiting examples of viral products include sindbis, VSV, oncoma, hepatitis A, channel cat fish virus, RSV, corona virus, FMDV, rabies, polio, reo virus, measles, and mumps.

Hormones also may be produced using the invention. Non-limiting examples of hormones include growth hormone (e.g., human growth hormone (hGH) and bovine growth hormone), growth factors, beta and gamma interferon, vascular endothelial growth factor (VEGF), somatostatin, platelet-derived growth factor (PDGF), follicle stimulating hormone (FSH), luteinizing hormone, human chorionic hormone, and erythropoietin.

Immunoregulators also may be produced. Non-limiting examples of immunoregulators include interferons (e.g., beta-interferon (for multiple sclerosis), alpha-interferon, and gamma-interferon) and interleukins (such as IL-2).

Metabolites (e.g., shikonin and paclitaxel) and fatty acids (i.e., including straight-chain (e.g., adipic acid, Azelaic acid, 2-hydroxy acids), branched-chain (e.g., 10-methyl octadecanoic acid and retinoic acid), ring-including fatty acids (e.g., coronaric acid and lipoic acid), and complex fatty acids (e.g., fatty acyl-CoA)) also may be produced.

The containers useful in the various embodiments of the invention may be of any size suitable for containing a liquid. For example, the container may have a volume between 1-40 L, 40-100 L, 100-200 L, 200-300 L, 300-500 L, 500-750 L, 750-1,000 L, 1,000-2,000 L, 2,000-5,000 L, or 5,000-10,000 L. In some instances, the container has a volume greater than 1 L, or in other instances, greater than 10 L, 20 L, 40 L, 100 L, 200 L, 500 L, or 1,000 L. Volumes greater than 10,000 L are also possible, but not desirable. Preferably, the container volume will range between about 1 L and 1000 L, and more preferably between about 5 L and 500 L, and even more preferably between 5 L and 200 L.

The components of the bioreactors and other devices described herein which come into contact with the liquid or products provided thereby desirably comprise biocompatible materials, more desirably biocompatible polymers, and are preferably sterilizable.

It should also be understood that many of the components described herein also are desirably flexible, e.g., the containers desirably comprise flexible biocompatible polymer containers (such as collapsible bags), with conduits which carry the fluids in and out of the container also desirably comprising such biocompatible polymers. The flexible material is further desirably one that is USP Class VI certified, e.g., silicone, polycarbonate, polyethylene, and polypropylene. Non-limiting examples of flexible materials include polymers such as polyethylene (e.g., linear low density polyethylene and ultra low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. If desired, portions of the flexible container may comprise a substantially rigid material such as a rigid polymer (e.g., high density polyethylene), metal, and/or glass.

Desirably the containers comprise biocompatible materials, more desirably biocompatible polymers. When collapsible containers are selected for use, the container may be supported by or may line an inner surface of a support structure, e.g., the platform, the latter having container-retaining sidewalls. However, the invention may be practiced using non-collapsible or rigid containers or conduits.

The containers may have any thickness suitable for retaining the liquid therewithin, and may be designed to have a certain resistance to puncturing during operation or while being handled. For example, the walls of a container may have a total thickness of less than or equal to 250 mils (1 mil is 25.4 micrometers), less than or equal to 200 mils, less than or equal to 100 mils, less than or equal to 70 mils (1 mil is 25.4 micrometers), less than or equal to 50 mils, less than or equal to 25 mils, less than or equal to 15 mils, or less than or equal to 10 mils. In certain embodiments, the container may include more than one layer of material that may be laminated together or otherwise attached to one another to impart certain properties to the container. For instance, one layer may be formed of a material that is substantially oxygen impermeable. Another layer may be formed of a material to impart strength to the container. Yet another layer may be included to impart chemical resistance to fluid that may be contained in the container.

It thus should be understood that a container may be formed of any suitable combinations of layers. The container (e.g., collapsible bag) may include, for example, 1 layer, greater than or equal to 2 layers, greater than or equal to 3 layers, or greater than equal to 5 layers of material(s). Each layer may have a thickness of, for example, less than or equal to 200 mils, less than or equal to 100 mils, less than or equal to 50 mils, less than or equal to 25 mils, less than or equal to 15 mils, less than or equal to 10 mils, less than or equal to 5 mils, or less than or equal to 3 mils, or combinations thereof.

In addition, the container preferably is seamless in order to improve its strength and avoid deposition of growing cells in the media.

All or portions of the container also are desirably translucent, or more desirably transparent, to allow viewing of contents inside the container. The latter is preferred when it is desirable to irradiate the liquid within the container.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A bioreactor suitable for housing a predetermined volume of liquid comprising nutrient medium and biological culture comprising:
(a) a container having at least one interior wall;
(b) at least one nutrient medium inlet;
(c) at least one outlet;
(d) at least one gas inlet;
(e) at least one gas outlet; and
(f) at least one cylindrical sparging filter attached to the at least one gas inlet,
wherein the sparging filter comprises a plurality of pores along its axis which permit gas to be emitted radially from the sparging filter into the liquid, wherein the diameter of the plurality of pores does not exceed about 50 μm, and wherein the orientation of the at least one sparging filter within the container provides for immersion of the plurality of pores within the liquid and substantially uniform distribution of emitted gas throughout the liquid.

2. The bioreactor according to claim 1, wherein the bioreactor comprises a plurality of the cylindrical sparging filters.

3. The bioreactor according to claim 2, wherein the axes of the plurality of cylindrical sparging filters are separated from one another by a distance of no more than about 1.5 feet.

4. The bioreactor according to claim 3, wherein the axes of the plurality of sparging filters are separated from one another by a distance of no more than about 1 foot.

5. The bioreactor according to claim 1, wherein the container is substantially cylindrical, and wherein the axis of the container is collinear with the axis of one of the cylindrical sparging filters.

6. The bioreactor according to claim 1, further comprising liquid in an amount sufficient to provide for immersion of the plurality of pores of the cylindrical sparging filter within the liquid.

7. The bioreactor according to claim 1, wherein the container is generally cylindrical, flexible, and the internal portion of the container is comprised of biocompatible material.

8. The bioreactor according to claim 7, wherein the axis of the at least one cylindrical sparging filter is located no more than about 1.5 feet from the container wall.

9. The bioreactor according to claim 8, wherein the axis of the at least one cylindrical sparging filter is located no more than about 1 feet from the container wall.

10. The bioreactor according to claim 7, further comprising an outer support for said flexible container.

11. The bioreactor according to claim 1, further comprising a plurality of sensors.

12. The bioreactor according to claim 1, further comprising sensor for detecting the temperature of the liquid within the container, and a means for controlling the temperature of the liquid.

13. The bioreactor according to claim 1, wherein the diameter of the plurality of pores does not exceed about 10 μm.

14. The bioreactor according to claim 1, wherein the axis of the at least one cylindrical sparging filter is located no more than about 2 feet from the container wall.

15. The bioreactor according to claim 1, wherein the container is generally a cylinder or a cuboid.

16. The bioreactor according to claim 1, further comprising two gas inlets, wherein each end of the cylindrical sparging filter is attached to a gas inlet.

17. The bioreactor according to claim 6, wherein the biological culture comprises mammalian cells or plant cells.

18. The bioreactor according to claim 6, wherein the biological culture comprises bacteria, yeast, hybrodomas or baculoviruses.

19. The bioreactor according to claim 1, further comprising microcarrier beads.

20. The bioreactor according to claim 19, wherein the microcarrier beads comprise one or more of silica, glass, dextran or polystyrene.

21. The bioreactor according to claim 1, wherein the interior of the container is free of mechanical agitation devices.

22. The bioreactor according to claim 21, wherein the bioreactor is stationary during operation.

23. The bioreactor according to claim 21, wherein the liquid is not agitated via mechanical devices during operation of the bioreactor.

24. The bioreactor according to claim 6, wherein the amount of liquid in the container during operation of the bioreactor exceeds about 60 vol. % of the container volume.

25. The bioreactor according to claim 24, wherein the amount of liquid in the container during operation of the bioreactor exceeds about 70 vol. % of the container volume.

26. The bioreactor according to claim 25, wherein the amount of liquid in the container during operation of the bioreactor exceeds about 80 vol. % of the container volume.

27. The bioreactor according to claim 26, wherein the amount of liquid in the container during operation of the bioreactor exceeds about 90 vol. % of the container volume.

28. The bioreactor according to claim 1, wherein the gas inlet is in fluid communication with a source of compressed air.

29. The bioreactor according to claim 1, further comprising at least one means of introducing nutrient media into the container.

30. The bioreactor according to claim 29, further comprising a perfusion means which includes a filter having a mean pore size diameter ranging from about 1 μm to about 100 μm.

31. The bioreactor according to claim 29, wherein the perfusion means is immersed within the liquid.

32. The bioreactor according to claim 29, wherein the perfusion means comprises a plurality of perfusion filters.

33. The bioreactor according to claim 32, wherein the perfusion filters comprise stainless steel.

34. The bioreactor according to claim 32, wherein the perfusion filters comprise a porous polymer material.

35. The bioreactor according to claim 32, wherein the perfusion filters comprise a porous ceramic material.

36. The bioreactor according to claim 32, wherein the perfusion filters comprise monolithic, single grade, aluminum oxide porous ceramic, and comprises a mean pore size ranging from about 6 μm to about 90 μm.

37. The bioreactor according to claim 32, wherein the perfusion filters comprise a porous cellulosic material.

38. The bioreactor according to claim 29, wherein said perfusion filter is located adjacent to a cylindrical sparging filter.

39. A method for producing a biological product from a predetermined volume of a liquid comprising nutrient medium and a biological culture comprising:
(a) providing a bioreactor suitable for housing a predetermined volume of liquid comprising nutrient medium and biological culture comprising:
(i) a container having at least one interior wall;
(ii) at least one nutrient medium inlet;
(iii) at least one outlet;
(iv) at least one gas inlet;
(v) at least one gas outlet; and
(vi) at least one cylindrical sparging filter attached to the at least one gas inlet
wherein the sparging filter comprises a plurality of pores along its axis which permit gas to be emitted radially from the sparging filter into the liquid, wherein the diameter of the plurality of pores does not exceed about 50 μm, and wherein the orientation of the at least one sparging filter within the container provides for immersion of the plurality of pores within the liquid and substantially uniform distribution of emitted gas throughout the liquid;
(b) introducing nutrient medium into the container;
(c) introducing biological culture into the container;
(d) passing gas through the sparging filter and into the liquid;
(e) detecting the density of biological culture in the liquid at predetermined time intervals; and
(f) removing the liquid and any biological product produced thereby from the container when the density of the biological culture in the liquid in the container reaches a predetermined value.

40. The method according to claim 39, wherein the biological culture comprises bacteria.

41. The method according to claim 39, wherein the biological culture comprises yeast.

42. The method according to claim 39, wherein the biological culture comprises baculovirus.

43. The method according to claim 39, wherein the biological culture comprises hybrodomas.

44. The method according to claim 39, wherein the gas is air.

45. The method according to claim 39, wherein the bioreactor is stationary during steps (d)-(f).

46. The method according to claim 39, wherein the liquid is not agitated via mechanical devices during steps (d)-(f).
